(19) United States
(12) Patent Application Publication
(10) Pub. No.: US 2026/0272748 A1
(43) Pub. Date: Sep. 17, 2026

(54) Portable self-contained emergency medical sanitary workstation

(71) Applicants: Irene Fernandez, Richmond, CA (US); Deborah Zerda-Andrews, San Rafael, CA (US)

(72) Inventors: Irene Fernandez, Richmond, CA (US); Deborah Zerda-Andrews, San Rafael, CA (US)

(21) Appl. No.: 19/079,424

(22) Filed: Mar. 13, 2025

(51) Int. Cl. A61G 10/00; A61J 1/10; A61J 1/16; A61M 5/14

(52) U.S. Cl. CPC A61G 10/005; A61J 1/10; A61J 1/16; A61M 5/1417

(57) ABSTRACT

Disclosed is a portable medical sanitary emergency workstation suitable for deployment in medically unsafe conditions that facilitates setup and use of IV bags or other injectables and medications. The workstation is essentially a carrying case-like enclosure when closed for portability, the case comprised of two main hingeably connected housings. The lower housing comprises a specialized sanitary workspace while the upper housing comprises support equipment storage, electrical power storage and control means, and pressurizing fan and air distribution means. Sanitary environment of the workspace is achieved by an expandable work surface cover, interior of which is pressurizable and accessible by attachable flexible gloves. Further included are additional sanitary and ease of use components necessary for the preparation and use of IV bags or other injectables or oral medications that can be filled quickly with on the spot required liquid medicines. This workstation would be used by medical professional following state laws.

### CROSS-REFERENCE TO RELATED APPLICATIONS

Not applicable.

### STATEMENT REGARDING FEDERALLY SPONSORED RESEARCH OR DEVELOPMENT

Not applicable.

### DESCRIPTION OF ATTACHED APPENDIX

Not applicable.

### BACKGROUND OF THE INVENTION

Currently, when an outdoor emergency medical event strikes, such as earthquake or major fire for example, trained medical personnel arrive with an assortment of emergency equipment including oxygen masks, body splints, pain medications, etc. However, there clearly is also a need for an in situ clean environment for mixing and preparing injectable drugs in these and similar hostile unsanitary environments. The present invention addresses this need with a portable self-contained sanitary workstation for the preparation of intravenous drip bags, i.e. IV bags, or other injectables or oral medications that can be filled quickly with on the spot required liquid medicines, and which can be easily transported to an inconvenient and hygienically hostile environment in emergency situations, such as the ones mentioned above.

A clean environment for mixing injectable drugs has been addressed by U.S. Pat. No. 10,675,220 granted to the present inventors, Irene Fernandez and Deborah Zerda-Andrews. However, the present invention provides a self-contained and sanitary IV bag filling workstation that can be carried by a person.

The present invention additionally addresses the need for organized storage easy access to items associated with the preparation and use of IV bags, such as a safe container for used needles, alcohol bottles or wipes. Additional features enable the self-contained in-situ use, and the present invention further provides for powering and lighting of the workstation without having to rely on external electrical power sources by including a renewable energy storage, and integrated solar power generation means.

### BRIEF SUMMARY OF THE INVENTION

In keeping with the foregoing, a portable self-contained medical emergency workstation providing a sanitary facility suitable for emergency use in outdoor and/or other medically unsafe environment that can be carried by a person like luggage or on a person's back and that facilitates the setup for preparation and use of IV bags, is solved with the present invention. The portable self-contained medical emergency workstation as described in the present patent application has been developed by the herein named inventors. The portable self-contained medical emergency workstation comprises two main components: an upper housing, also referred to as the second main housing, and a lower housing, also referred to as the first main housing, each typically but not necessarily, being essentially cuboids. The upper and the lower housings can be securely joined with latches, such as clasps or similar devices while also being pivotally connected, thus creating essentially a carrying case-like enclosure when joined for portability. The portable self-contained medical emergency workstation further comprises additional extendable or attachable/detachable medical work components as required in a specific adverse situation.

The focus of the present invention is to provide a portable self-contained medical emergency workstation for in situ filling of IV bags and mixing medicines. The upper housing includes compartments for storing medical items needed for IV related activities in a typical embodiment. The lower housing includes a specialized work surface for preparing and manipulating an IV bag cradle so that an IV bag can be filled with injectable medications. An included collapsible transparent housing covering the work surface creates a sanitary workspace environment that is dust-free and dirt-free, suitable for filling and manipulating IV bags or injectable medications. A gloved access included in the front of the collapsible transparent housing enables a person to prepare and manipulate items in the housing in a sanitary way. The transparent housing disclosed in the preferred embodiment is a foldable one, comprised of a plurality of panels. In an alternate embodiment, the transparent housing is inflatable with the fan and ducts disposed in the upper housing. Both transparent housing embodiments are collapsible to enable the workstation to be closed.

The fan and at least one air duct included in the upper housing are used to blow filteredⁱ air into the transparent housing to further ensure the workspace to be dust-free and dirt-free. Additionally, an included extendable pole can be extended from the top of the upper housing providing capability to suspend additional items, especially IV bags, when the workstation is open. Furthermore, one or more drawers are provided, located under the work surface for storing other essential medical items. The portable self-contained medical emergency workstation also comprises individually adjustable legsⁱⁱ attached to the lower housing that can be swung out from the underside of the workstation to support the workstation with the work surface at a stable working height even on uneven ground.

The upper housing also includes an energy compartment containing a renewable energy storage apparatus and electric power distribution and control devices. In one embodiment the renewable energy storage comprises rechargeable batteries. Solar panels are disposed on the larger outer surface of the upper housing for keeping the energy storage rechargeable batteries charged or at least extend the time electric power is available without externally added energy source or sources.

Other embodiments of renewable energy storage that can be used are supercapacitors, fuel cells, thermal energy storage with thermoelectric generators and even flywheel energy storage. The solar panels and rechargeable batteries are the most widely used and therefore used in the preferred embodiment.

Electric power outlets are accessible on the outside of the upper housing to power external devices. Electric lighting is included in the upper housing to facilitate work at night or in unlit environments.

Means for attaching a pair of shoulder straps are provided on the upper housing to enable a person to carry the portable self-contained medical emergency workstation on the person's back.

This summary is provided to introduce preferred embodiments of the invention, a portable self-contained medical emergency workstation, geared toward filling, manipulating and and preparing injectable medications. The disclosed portable self-contained medical emergency workstation enclosure that can transform into a sanitary, i.e. sanitary, portable work station for manipulating IV bags and/or other activities requiring a sanitary enclosed environment; said enclosure comprised of at least two main parts that when connected by hingeable mechanisms form an essentially suitcase-like prism capable of being held by an integrated carry handle; the main parts removably retaining medical supplies associated with the handling of IV bags, telescopingly extendable IV support pole for hanging IV bags, a work platform, a foldable transparent housing that can enclose the work platform, the foldable transparent housing including a pair of apertures having inward facing gloves for manipulating items within said transparent housing, a set of drawers and cubicles for holding additional medical items.

This summary introduces a simplified selection of concepts of providing a portable self-contained medical emergency workstation that will further be described in a detailed descriptions hereinafter. This summary is not intended to identify essential features of the invention, nor is it intended to limit the scope of the present invention.

This summary is not intended to identify key and essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a perspective view of the portable self-contained medical emergency workstation in a closed state, i.e. transport, state, showing the external sides that include folding legs and power outlets.

FIG. 2 is a perspective view of the portable self-contained medical emergency workstation in a closed state showing the side of the upper housing, also referred to as the second main housing, that includes a solar panel or panels.

FIG. 3 is a perspective view of the portable self-contained medical emergency workstation enclosure in the open position depicting the transparent housing with the foldable panels embodiment with the panels folded down.

FIG. 4 is a perspective view of the portable self-contained medical emergency workstation housing with the foldable panels embodiment with the side panels being lifted.

FIG. 5 is a perspective view of the portable self-contained medical emergency workstation housing in a ready to use state including the fully assembled transparent housing and the gloved access clearly visible.

FIG. 6 is a perspective view of the portable self-contained medical emergency workstation showing the IV bag support pole in place on the top of the fully deployed portable self-contained medical emergency workstation enclosure.

FIG. 7 is a perspective view showing the underside of the lower housing, also referred to as the first main housing, and the back of the upper housing of the fully deployed portable self-contained medical emergency workstation.

FIG. 8 is a side view of the IV support pole in its extended position.

FIG. 9 is a front view of a person wearing the portable self-contained medical emergency workstation enclosure on the person's back.

FIG. 10 is a side view of the portable self-contained medical emergency workstation enclosure showing externally accessible power ports, battery level indicators and an attached carrying strap.

FIG. 11 is a perspective view of the portable self-contained medical emergency workstation in the ready to be transported state showing the side of the workstation that includes the solar panel.

FIG. 12 is a perspective view of the portable self-contained medical emergency workstation in the use position with the transparent housing removed and an additional panel placed on top of the drawer for an additional work surface.

FIG. 13 is a partial perspective view showing the movable panels for creating an air lock access to the auxiliary enclosure.

FIG. 14 is a perspective view of the underside of the portable self-contained medical emergency workstation when in the closed position.

### DETAILED DESCRIPTION OF THE PREFERRED EMBODIMENTS

Detailed descriptions of the preferred embodiment of the portable self-contained medical emergency workstation are provided herein. It is to be understood, however, that the present portable self-contained medical emergency workstation may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

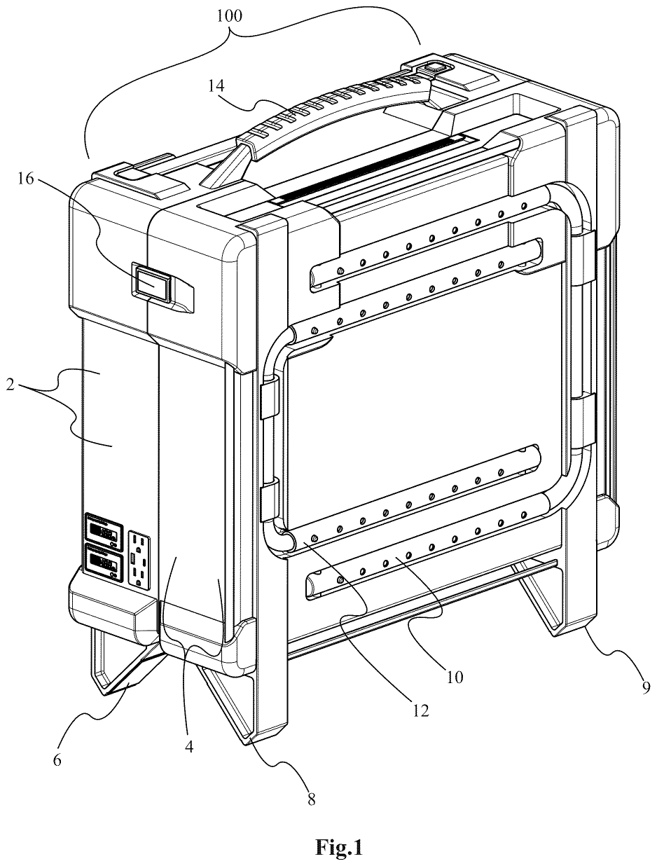

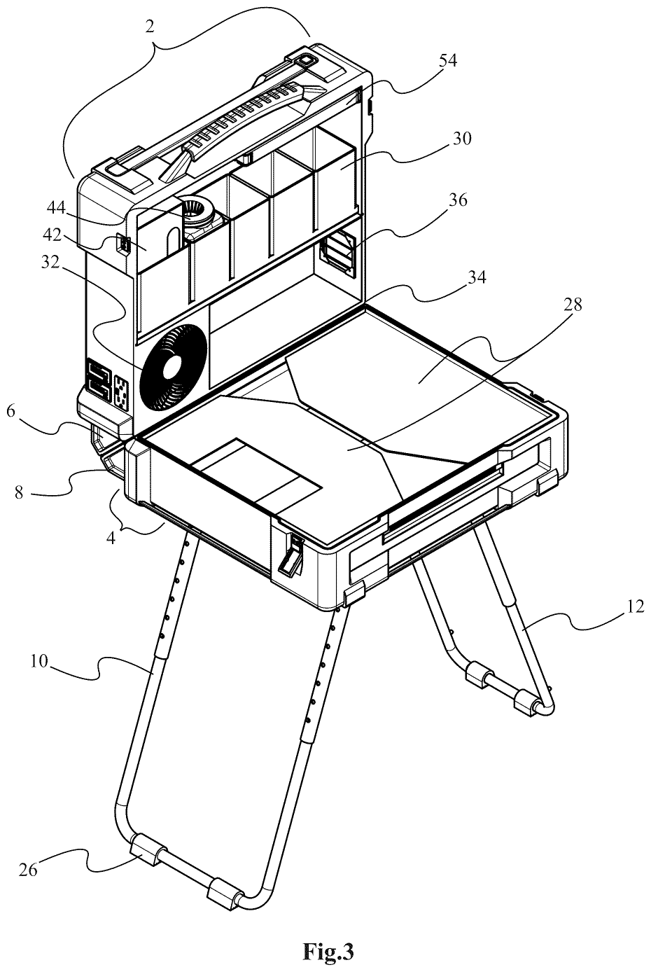

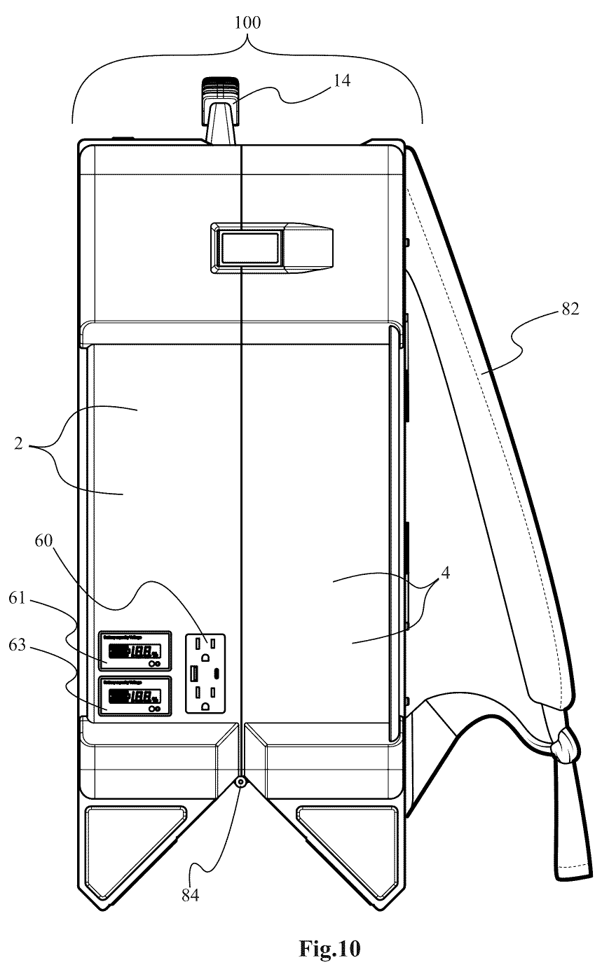

Referring now to FIG. 1 we see a perspective view of the portable self-contained medical emergency workstation 100 primarily showing the lower housing 4. The workstation is essentially a carrying case-like cuboid case when closed for portability. The workstation comprises an upper housing 2 and a lower housing 4, the upper and lower housings being connected by a hinge mechanism 84, the hinge mechanism can be a piano hinge, or a plurality of short hinges or other flexible device capable of being folded while also holding the two housings together as shown in FIG. 3 and in FIG. 10. The upper and lower housings are releasably held together by latches such as clasps 16 shown in the instant embodiment. Feet 6, 8, 9, and 11, not visible on FIG. 1, help to keep the workstation 100 off the ground when in the closed carry position and also help keep the upper housing 2 at a right angle to the lower housing 4 in the open deployed position, as shown in FIG. 3. Foldout legs 10 and 12 are shown nested into the outer surface of the lower housing 4. A handle 14 allows a person to easily carry the workstation 100 from one location to another.

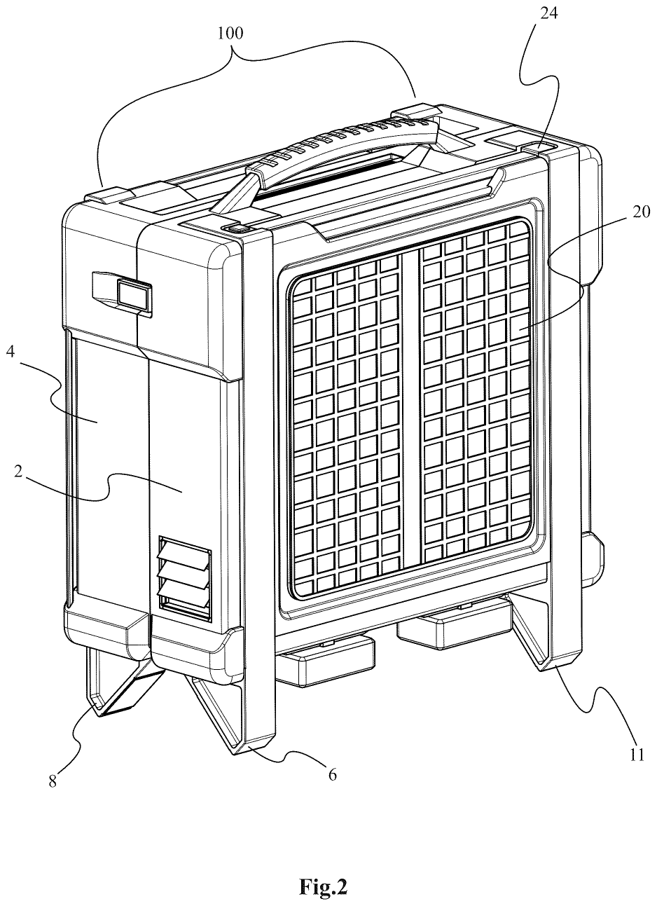

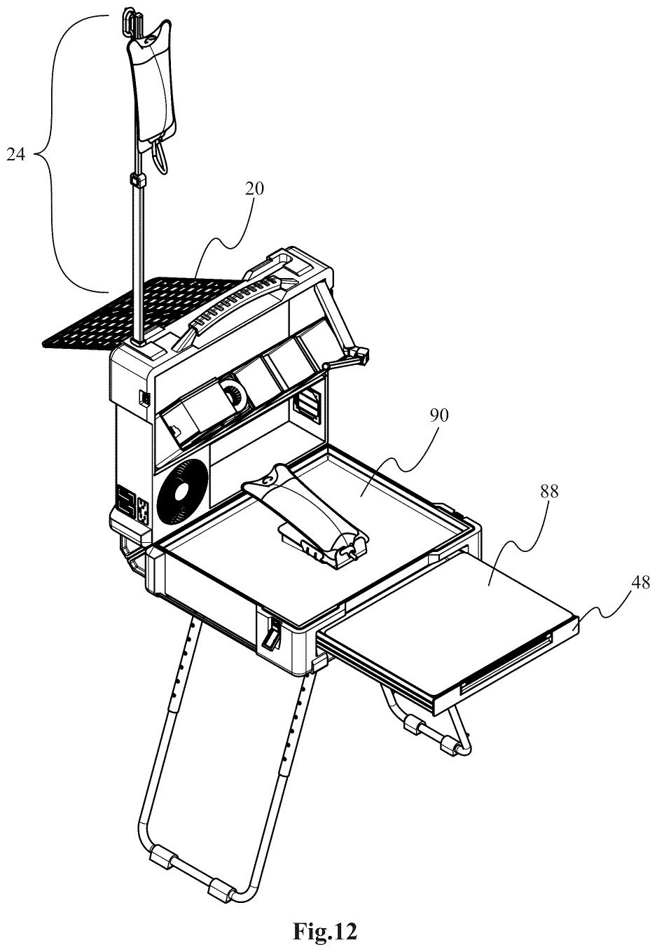

FIG. 2 is a perspective view of the workstation 100 primarily showing upper housing 2 which has a solar panel 20 attached to its outer surface. The solar panel 20 is connected with a power inverter supplying power to a battery, and other power control and distribution modules, not shown, inside upper housing 2. An inverter, not shown, converts the solar generated power into a format required for powering external plug-in electrical appliances, typically 120 and 240 VAC and to a format required for charging or powering smart phones and other portable electronic devices, typically 5 volts DC. The other power control and distribution modules primarily power the air fan and lights within the workstation. IV hanger pole 24 is nested into the upper housing 2 and can be raised to support an IV bag as shown in FIG. 12.

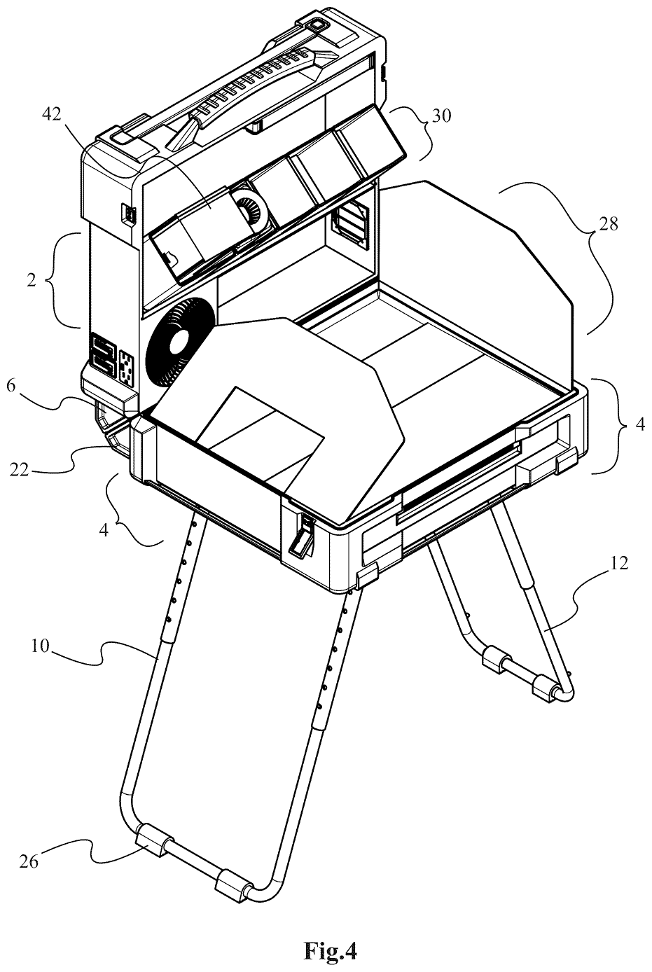

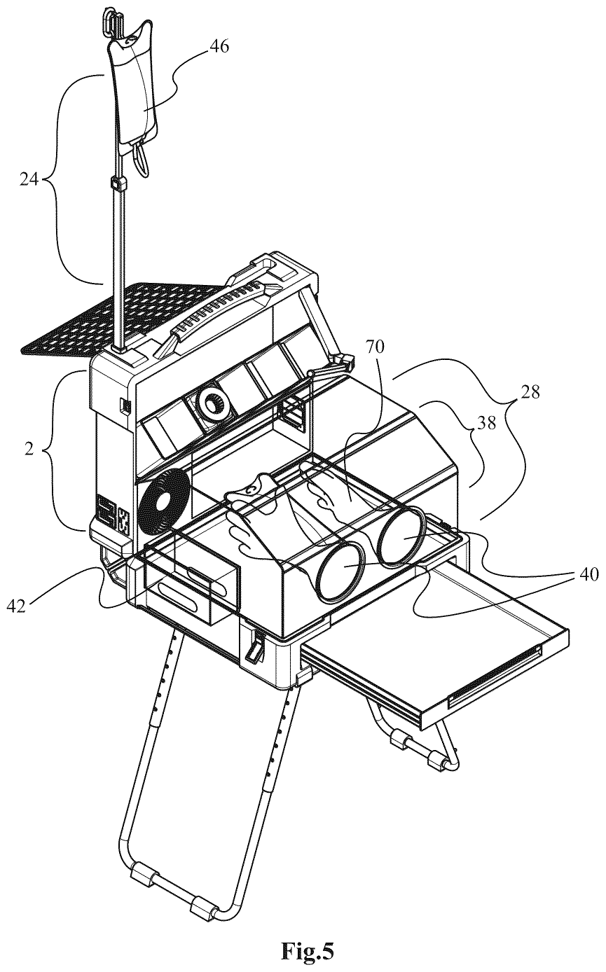

FIG. 3 is a perspective view of the workstation 100 in the open position with the upper housing 2 situated at a ninety-degree angle from the lower housing 4. Legs 10 and 12, located in enclosure half 4 are deployed to allow the workstation to be at a height that can allow a person to use the workstation without bending down. The legs 10, 12 each are comprised of two telescoping parts, with holes for example as shown here—there are other well known ways to implement adjustable legs so that they can be adjusted to various heights, e.g. three legs arranged as a tripod, a frequently used method of using various devices required stabilization and/or leveling when used on an uneven surface. Leg bottoms include non-slip feet 26 to further stabilize the entire workstation 100. Folded transparent panels 28 can be lifted as shown in FIGS. 4 and 5 to form a dirt and dust proof enclosure. Fan 32 blows filtered air into the enclosure 28 to keep it pressurized, thusly free of dust. The upper housing 2 includes compartments 30 for storing items associated with the deployment of IV bags, and including used needle storage 43, and also other items such as a bottle or bottles of 70% isopropyl alcohol or disinfecting agents, a plurality of wipes and bandages as well as a place to hold an auxiliary sanitary container 42 that can be removed and placed on the side of the main foldable transparent housing panel 28 as shown in FIG. 5 to create a clean transfer station for items that will be introduced into the foldable transparent housing when unfolded. An LED light 54 can be swung out, as shown in FIG. 12 to help illuminate the work surface 90 as shown in FIG. 12.

FIG. 4 shows a perspective view of the workstation 100 with the transparent panels 28 partially lifted. The compartments 30 have been pivoted out from the upper housing 2 to allow for easy access to the contents of the compartments 30.

FIG. 5 is a perspective view of the workstation 100 showing the auxiliary sanitary container 42 being added to the side of the transparent housing panel 28. The working surface 90 is made of ferrous metal material having a cleanable impervious surface, typically powder-coated or with another cleanable impervious topcoat. Flexible gloves 70 are attached to apertures 40 to allow a person to manipulate items inside the transparent housing while maintaining a dust-free and dirt-free environment. Drawer 48 is shown pulled out from the lower housing 4. Drawer 48 can retain additional smaller items associated with mixing injectable activities including small cups, syringes and needles.

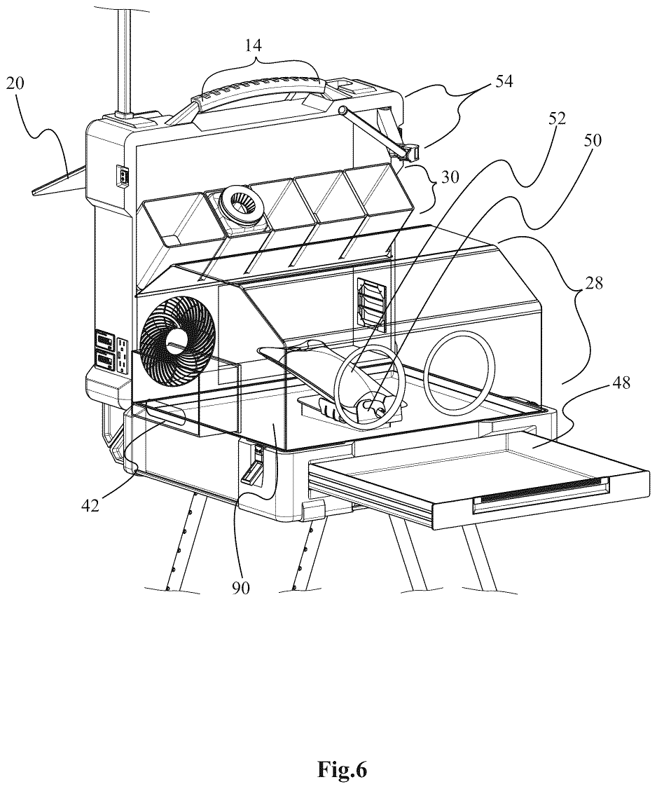

FIG. 6 shows a clearer view of work surface 90 as well as IV bag holder 50 and IV bag 52. IV bag holder 50 has magnets built into its bottom surface to engage the ferrous metal work surface 90 thereby holding it in place during use. IV bag 52 can be seen being held in place by IV bag holder 50. The IV bag holder 50 can be configured in various configuration, e.g. U.S. Pat. No. 10,675,220 by the instant inventors depicts a magnetic cradle shaped IV bag tip holder.

Solar panel 20 can also be seen swung out to allow it to have a more direct angle in relation to the sun.

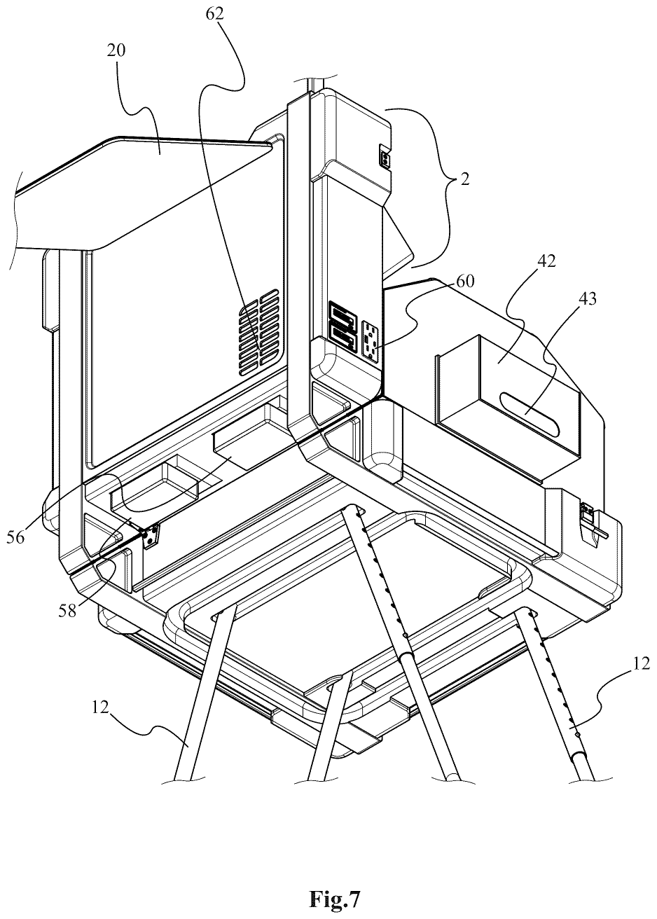

FIG. 7 is a perspective view of the underside of the medical enclosure of workstation 100. Rechargeable and replaceable batteries 56 are charged by the solar panel 20 or can be charged by conventional means using a charging station that is plugged into an external power source, The batteries 56 are capable of powering LED light 54 or any other item that needs to be charged via USB ports or power outlets 60 located on the outside wall of upper housing 2. Auxiliary sanitary container 42 is clearly visible which includes a recess 43 for lifting the front panel. The recess 43 is filled with solid material so that no dirt or dust can enter through the lifting area. Filtering air entrance vent 62 allows air to be drawn into upper housing 2 where it is filtered and then pushed through grill 36 as shown in FIG. 3. Solar panel 20 is shown in a swung out position to gain greater access to the sun.

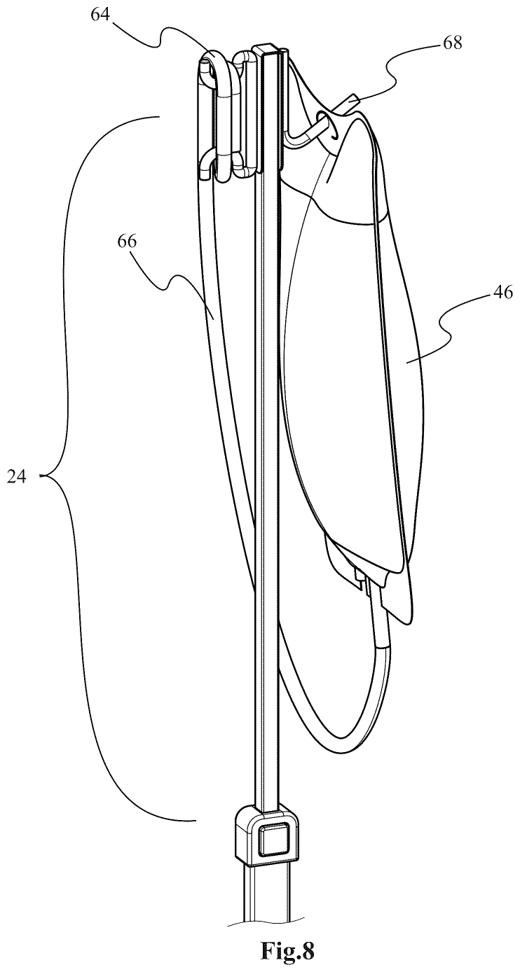

FIG. 8 is a detailed side view of the IV support pole assembly 24 which includes a hook 68 for holding a standard IV bag 46 and an additional pair of hooks 64 for wrapping excess tubing 66. This configuration allows the user to purge the tubing of any excess air before being injected into a patient.

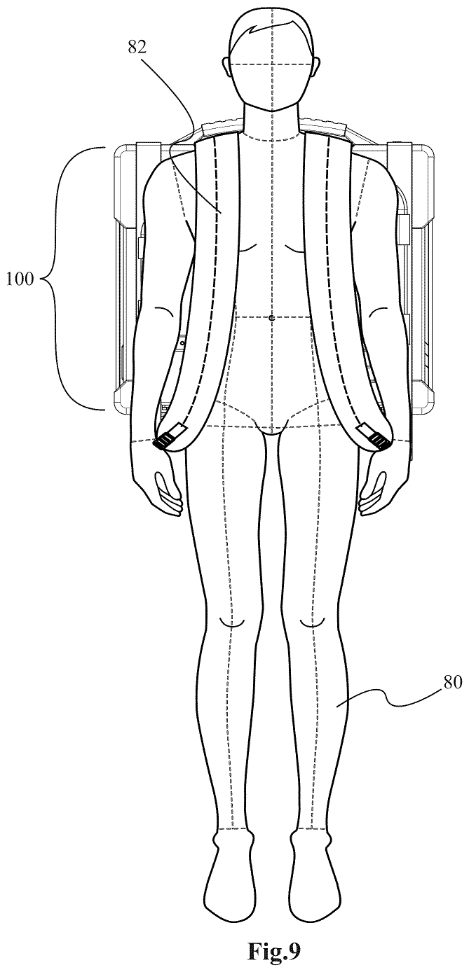

FIG. 9 is a front view of a person 12 wearing the emergency medical workstation 100 on his or her back via straps 82. In this way, a person can transport the medical enclosure 100 while traveling long distances or when there is a need to carry other items in the person's hands.

FIG. 10 is a side view of the workstation 100 clearly showing electric power outlets 60 as well as battery strength indicators 61, 63 located on the side wall of the upper housing 2. Strap 82 is also clearly shown attached to enclosure half 4. Handle 14 is also clearly shown.

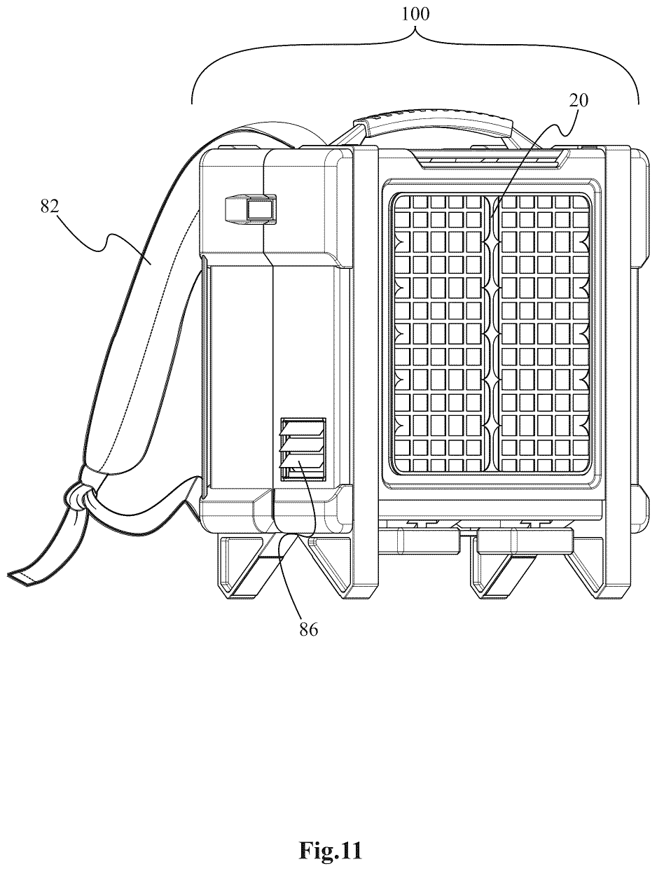

FIG. 11 is a rear view of the workstation 100 clearly showing solar panel 20 in its stored position. Air exit vent 86 is also clearly visible.

FIG. 12 is a perspective view of the workstation 100 in use without the clear transparent housing 28 being present. There may be times, perhaps during indoor use, where the enclosure 28 will not be necessary. In addition, more working area can be gained by adding a panel 88 to the top surface of drawer 48 thereby almost doubling the working surface when combined with main working surface 90.

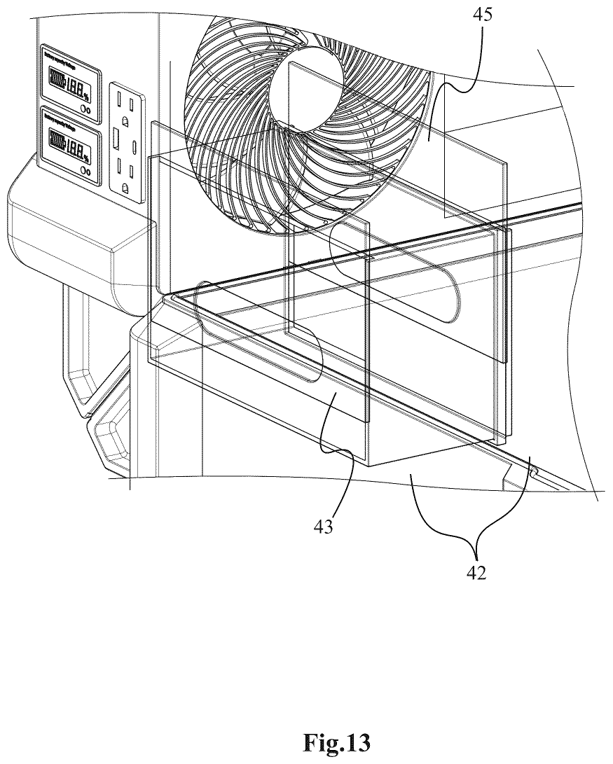

FIG. 13 is a partial perspective view clearly showing how an outer panel 43 can be lifted to insert an item into an auxiliary sanitary container enclosure 42 and then panel 43 can be lowered and inside panel 45 can be raised by a gloved hand within the main enclosure 28 thereby insuring that no dust or dirt can directly enter the main enclosure 28.

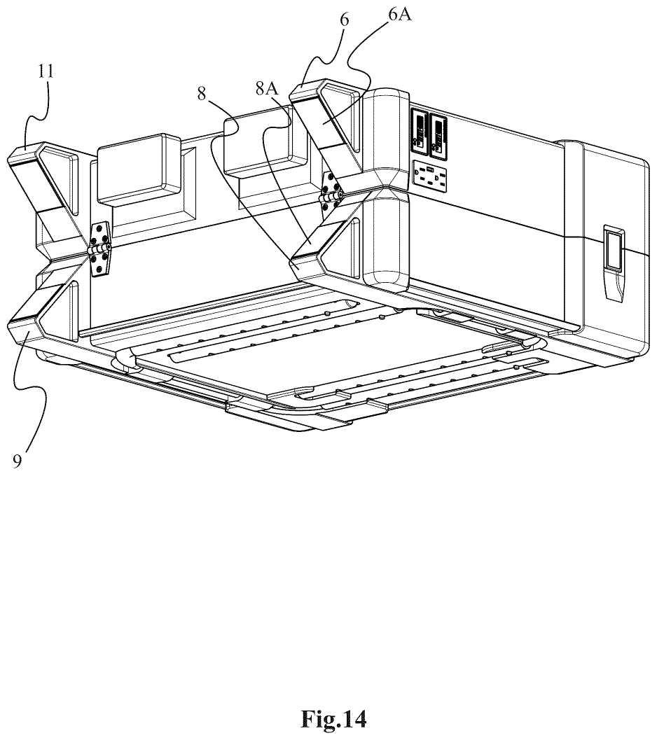

FIG. 14 is a view of the workstation 100 that shows the angled feet 6,8,9,11 which include magnets 6A, 8A so that when the housings 2, 4 are opened, the magnets 6A, 8A connect with each other insuring that the housings 2, 4 remain in a fixed position ninety degrees to each other until the user decides to close the workstation by overcoming the magnetic force imposed by the magnets 6A and 8A.

Additional electric power control and distribution subsystems for charging, i.e. resupplying, the rechargeable batteries, converting external electric power for direct distribution within the workstation and for charging the rechargeable batteries and other electrical and mechanical components are located within the enclosure, however they are standard and commonly known components and therefore not shown within the drawings that relate to the workstation. Parameters of electric power accessible through input and/or output external connections depend on the location where the medical workstation is being used, thus making the portable self-contained medical emergency workstation usable anywhere in the world.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

In the claims, the word ‘comprising’ does not exclude the presence of other elements or steps than those listed in a claim. Furthermore, the terms “a” or “an,” as used herein, are defined as “one, or more than one.” Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are arbitrarily used to distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

### REFERENCES VS FIGURE NUMBERS MATRIX

The matrix of references is included in the instant document for the Examiner's convenience and it is not intended to limit the hereinabove text set forth; the text set forth hereinabove is to be used in case of discrepancies.

| | FIG. | FIG. | FIG. | FIG. | FIG. | FIG. | FIG. | FIG. | FIG. | FIG. | FIG. | FIG. | FIG. | FIG. | |
|---|---|---|---|---|---|---|---|---|---|---|---|---|---|---|---|
| Reference | 1 | 2 | 3 | 4 | 5 | 6 | 7 | 8 | 9 | 10 | 11 | 12 | 13 | 14 | Description |
| 2 | x | x | x | x | x | x | x | | x | x | | | | | Upper housing |
| 4 | x | x | x | x | | | | | | x | | | | | Lower housing |
| 6 | x | x | x | x | | | | | | | | | | x | foot |
| 6A | | | | | | | | | | | | | | x | magnets |
| 8A | | | | | | | | | | | | | | x | magnets |
| 8 | x | x | x | | | | | | | | | | | x | foot |
| 9 | x | | | | | | | | | | | | | x | foot |
| 10 | x | | x | x | | | | | | | | | | | fold out leg |
| 11 | | x | | | | | | | | | | | | x | foot |
| 12 | x | | x | x | | | | | x | | | | | | fold out leg |
| 14 | x | | | | | | | | | x | | | | | carrying handle |
| 16 | x | | | | | | | | | | | | | | clasps |
| 20 | | x | | | | x | x | | | | x | x | | | solar panel |
| 24 | | x | | | x | | | x | | | | x | | | IV hanger pole |
| 26 | | | | x | | | | | | | | | | | non-slip feet |
| 28 | | | x | x | x | x | | | | | | | | | foldable transparent |
| | | | | | | | | | | | | | | | enclosure panels |
| 30 | | | x | x | | x | | | | | | | | | storage compartments |
| 32 | | | x | | | | | | | | | | | | fan |
| 36 | | | x | | | | | | | | | | | | air intake grill |
| 40 | | | | | x | | | | | | | | | | glove attachment |
| | | | | | | | | | | | | | | | appertures |
| 42 | | | x | x | x | x | x | | | | | | x | | removable auxiliary |
| | | | | | | | | | | | | | | | enclosure |
| 43 | | | x | | | | x | | | | | | x | | used needles storage |
| 45 | | | | | | | | | | | | | x | | an inside panel |
| 46 | | | | | x | | | x | | | | | | | an IV bag |
| 48 | | | | | | x | | | | | | x | | | drawer |
| 50 | | | | | | x | | | | | | | | | IV bag holder |
| 52 | | | | | | x | | | | | | | | | IV bag |
| 54 | | | x | | | x | | | | | | x | | | LED light |
| 56 | | | | | | | x | | | | | | | | rechargable & |
| | | | | | | | | | | | | | | | replacable batteries |
| 60 | | | | | | | x | | | x | | | | | power outlets |
| 61 | | | | | | | | | | x | | | | | battery strength indicator |
| 62 | | | | | | | x | | | | | | | | upper housing air vents |
| 63 | | | | | | | | | | x | | | | | battery strength indicator |
| 64 | | | | | | | | x | | | | | | | pair of hooks |
| 66 | | | | | | | | x | | | | | | | tubing |
| 68 | | | | | | | | x | | | | | | | IV hook |
| 70 | | | | | x | | | | | | | | | | flexible gloves |
| 82 | | | | | | | | | x | x | x | | | | carrying straps |
| 84 | | | x | | | | | | | x | | | | | upper & lower housing |
| | | | | | | | | | | | | | | | connecting hinges |
| 86 | | | | | | | | | | | x | | | | air exit vent |
| 88 | | | | | | | | | | | | x | | | extra panel |
| 90 | | | | | x | x | | | | | | x | | | work surface |
| 100 | x | x | | | | | | | x | x | x | | | | The workstation |

What is claimed is:

1. A portable self-contained medical emergency workstation comprising a first main housing and a second main housing, the first main housing and the second main housing each being essentially of a three-dimensional shape, each housing containing equipment required for sanitary filling and manipulating intravenous bags and mixing injectable drugs, wherein each main housing comprising: a top, a bottom, the top and the bottom being parallel essentially planar surfaces, each planar surface having a perimeter, and at least one side disposed between the top and the bottom surfaces perimeters, the first main housing top and the second main housing bottom being congruent, wherein furthermore:
each housing having a front and a back, the first and the second main housings being hingeably connected to each other by a hinge mechanism disposed in the backs of the first and of the second main housings;
the second main housing comprising at least two stable positions: a first stable position and a second stable position, wherein the first stable position is defined by the second main housing bottom being in intimate contact with the congruent first main housing top thus creating a single case comprised of the two main housings held together by the hinge mechanism and by a securement mechanism, wherein the second stable position is defined by the second main housing being pivoted up via the hinge mechanism to be orthogonal to the first main housing whereby enabling access to the first main housing top surface;
the portable self-contained medical emergency workstation further comprising a collapsible transparent essentially sealed enclosure disposed on the top of the first main housing, the transparent essentially sealed enclosure having a flat state and an erected state;
the transparent essentially sealed enclosure forming a sanitary workspace disposed over the first main housing top when the transparent sealed enclosure is in the erected state, the transparent sealed enclosure enclosure further comprising at least two glove attachment apertures, enabling a person to handle items within the transparent sealed enclosure while the handled items remain fully enclosed within the transparent sealed sanitary enclosure separated from a non-sanitary environment outside of the transparent sealed enclosure;
the transparent essentially sealed enclosure enclosure further comprising at least one pressurizing air intake aperture;
the first main housing further comprising a plurality of rotatably attached telescopingly extendable legs disposed in the first main housing bottom;
additional portable self-contained medical emergency workstation components disposed within the second main housing comprise:
a plurality of pivotally accessible removable compartments wherein at least one of the compartments comprising a used needles storage, wherein furthermore at least one of the compartments is a removable auxiliary sanitary container;
a renewable energy storage apparatus;
an energy storage and distribution system;
an energy storage resupply system;
an energy storage and resupply system: control, power distribution and status indication apparatus;
a telescoping intravenous bag hanger pole, the hanger pole being disposed within the second main housing when fully compacted and being capable of suspendigly holding an intravenous bag above the second main housing when extended and when the second main housing is in the second stable position;
at least one lighting device; and
at least one transparent essentially sealed enclosure pressurizing filtered air fan.

2. The portable self-contained medical emergency workstation of claim 1 wherein the energy storage apparatus comprising rechargeable batteries, wherein furthermore the energy storage resupply system comprising at least one rotatable solar panel.

3. The portable self-contained medical emergency workstation of claim 1, wherein the lighting device is a lamp comprising a plurality of light emitting diodes.

4. The portable self-contained medical emergency workstation of claim 1, wherein the first main housing further comprising:
an intravenous bag holder, disposed within the intravenous bag transparent filling enclosure on the first main housing top panel; and
at least one drawer comprising a top that serves as an additional work surface.

5. The portable self-contained medical emergency workstation of claim 1 wherein the IV hanger pole comprises at least one hook for suspending an IV bag and associated tubing.

6. The portable self-contained medical emergency workstation of claim 1 wherein disposed on the bottom of the first main housing is a plurality of shoulder straps and wherein furthermore disposed on the front of the second main housing is a carrying handle, whereby the shoulder straps and the carrying handle enabling the portable self-contained medical emergency workstation to be carried by a single person.

7. The portable self-contained medical emergency workstation of claim 1, wherein the transparent essentially sealed enclosure comprises a plurality of transparent panels, wherein the plurality of the panels capable of being in a folded position or in an erected position, the transparent essentially sealed enclosure being essentially flatly disposed on the first main housing top, wherein the transparent essentially sealed enclosure forming a sanitary enclosure disposed over the first main housing top panel when in the erected position, the plurality of the transparent panels when in the erected position comprising a front panel and a rear panel when in the erected position, wherein the front panel comprising the glove attachment apertures, wherein the rear panel comprising the pressurizing air intake apertures.

8. The portable self-contained medical emergency workstation of claim 1, wherein the pivot mechanism comprising at least one hinge.

9. The portable self-contained medical emergency workstation of claim 1 wherein the first main housing and the second main housing each being essentially of a three-dimensional box shape comprising: at least one side panel, a front panel, a back panel, a top panel and a bottom panel, wherein each panel being orthogonal to each adjacent panel, wherein the first main housing top panel and the second main housing bottom panel being congruent.

10. The portable self-contained medical emergency workstation of claim 9, comprising angled feet disposed on the back panel of the first main housing and on the back panel of the second main housing, the angled feet comprising magnets, the magnets keeping the first main housing and the second main housing stable when the two main housings are in the second stable position.

11. The portable self-contained medical emergency workstation of claim 9, wherein externally accessible electric power outlets being disposed on the second main housing side panel.

12. The portable self-contained medical emergency workstation of claim 1, comprising at least one air vent disposed on the second main housing.

13. The portable self-contained medical emergency workstation of claim 9, wherein the first main housing additionally comprising an access mechanism for holding the removable auxiliary sanitary container, wherein the access mechanism comprises a slidable inside panel operable from within the transparent filling housing and a slidable outside panel making the auxiliary removable sanitary container accessible from within the erected transparent essentially sealed enclosure without exposing the removable sanitary container to a non-sanitary environment outside of the transparent sealed enclosure.
